(19) United States
(12) Patent Application Publication
(10) Pub. No.: US 2024/0065638 A1
(43) Pub. Date: Feb. 29, 2024

(54) DARK BLOOD CARDIAC MAGNETIC RESONANCE IMAGING WITH INTERRUPTED PARTIALLY UNBALANCED TIME-REVERSED STEADY-STATE FREE PRECESSION PULSE SEQUENCES

(71) Applicant: NOTHSHORE UNIVERSITY HEALTHSYSTEM, Evanston, IL (US)

(72) Inventor: ROBERT R. EDELMAN, Highland Park, IL (US)

(21) Appl. No.: 17/895,521

(22) Filed: Aug. 25, 2022

(51) Int. Cl. A61B 5/00; A61B 5/055; G01R 33/567

(52) U.S. Cl. CPC A61B 5/7292; A61B 5/055; G01R 33/5673; A61B 5/704

(57) ABSTRACT

A dark blood magnetic resonance imaging (MRI) imaging technique utilizes a time-reversed steady-state free precession (SSFP) pulse sequence, in which magnetic field gradients are unbalanced along at least one gradient axis, but balanced along at least one of the other gradient axes. The pulse sequence can include interrupted shots, in which subsequent shots or repetitions of the pulse sequence are not continuous in time. For example, the pulse sequence can be gated based on cardiac signals, respiratory signals, or navigator data, or may be otherwise discontinuous over time.

### BACKGROUND

Rapid gradient-echo magnetic resonance imaging (“MRI”) pulse sequences (i.e., where the sequence repetition time (“TR”) is short compared with the tissue T1 and T2 relaxation times) fall into three general categories: radio frequency (“RF”) spoiled, balanced steady-state free precession (“SSFP”), and unbalanced gradient-spoiled SSFP. Whereas RF spoiled sequences discard residual transverse magnetization at the end of each sequence repetition, gradient-spoiled SSFP sequences reuse it, resulting in high signal from tissues that have a large T2/T1 ratio.

With balanced SSFP (bSSFP, also called trueFISP, bFFE, or FIESTA) all three imaging gradients are “balanced,” meaning they have net zero area over each sequence repetition. Flowing intravascular spins appear bright, which makes the technique particularly useful for cardiac cine imaging and non-contrast MR angiography. Conversely, with unbalanced gradient-spoiled SSFP techniques, which include SSFP-FID (e.g., FISP, GRASS, FFE) and SSFP-echo (e.g., PSIF, GRASS, T2-FFE), the slice-select and readout gradients are unbalanced, meaning that each gradient has a net non-zero area over each sequence repetition.

With SSFP-FID, a free induction decay (“FID”) signal is collected in each sequence repetition after which one or more additional spoiler gradients may be applied. With SSFP-echo, the pulse sequence structure is essentially reversed from SSFP-FID. As a result, the FID signal is dephased. Instead, a spin-echo signal is refocused from magnetization generated by an RF pulse that was applied during the prior sequence repetition. Compared with SSFP-FID, SSFP-echo is more T2- and diffusion-weighted. Both SSFP-FID and SSFP-echo sequences dephase flowing spins, resulting in dark blood images. These techniques have been applied for dark blood imaging of the aorta, carotid arteries, and peripheral arteries, but are too motion-sensitive to be reliably used for dark blood imaging of the heart.

An ECG-gated SSFP-FID technique that is unbalanced in the slice-select and readout directions has shown promise for dark blood imaging of the aorta, but remains too motion sensitive to be used reliably for the heart, particularly in patients with fast heart rates.

### SUMMARY OF THE DISCLOSURE

The present disclosure addresses the aforementioned drawbacks by providing a method for producing an image of a subject using a magnetic resonance imaging (MRI) system, in which the image has a dark blood image contrast. The method includes acquiring magnetic resonance data from a subject by controlling the MRI system to perform a time-reversed steady-state free precession (SSFP) pulse sequence. The time-reversed SSFP pulse sequence includes, in each of a plurality of interrupted shots: applying a radio frequency (RF) excitation pulse to a prescribed imaging location; applying unbalanced magnetic field gradients along at least a first spatial encoding direction; and applying balanced magnetic field gradients along at least a second spatial encoding direction. An image is reconstructed from the magnetic resonance data, where the image depicts a dark blood image contrast in which blood is depicted darker than other tissues.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a pulse sequence diagram of an example partially unbalanced, time-reversed steady-state free precession (“SSFP”) pulse sequence, which may be referred to as a partially unbalanced SSFP-echo pulse sequence, in which gradients are unbalanced along at least the slice select (or partition encoding) gradient axis, but are balanced along at least the readout gradient axis.

FIG. 2 is a pulse sequence diagram of an example partially unbalanced SSFP-echo pulse sequence, in which gradients are unbalanced along at least the readout gradient axis, but are balanced along at least the slice select (or partition encoding) gradient axis.

FIG. 3 are example images acquired using partially unbalanced SSFP-echo pulse sequences with and without inversion recovery preparation and gadolinium enhancement, as compared with an image acquired with a post-gadolinium, IR-prepared balanced SSFP pulse sequence.

FIG. 4 shows example images acquired with a fully unbalanced SSFP-FID pulse sequence (top row) at various flip angles as compared to images acquired with a partially unbalanced SSFP-echo pulse sequence (bottom row) using the same flip angles.

FIG. 5 shows various multi-contrast images acquired with partially unbalanced SSFP-echo pulse sequences, as compared to an image acquired with a volumetric interpolated breath-hold examination (“VIBE”) pulse sequence.

FIG. 6 shows an example image obtained with a partially unbalanced SSFP-echo pulse sequence without prep pulses (left) and an images obtained with a partially unbalanced SSFP-echo pulse sequence using a T2-preparation (right).

FIG. 7 is a block diagram of an example MRI system that can implement the methods described in the present disclosure.

### DETAILED DESCRIPTION

Described here are systems and methods for magnetic resonance imaging that acquire images in which blood appears dark relative to other tissues in the images. These so-called “dark blood” imaging techniques are advantageous for distinguishing lesions, scar tissue, or other tissues from nearby vessels or cardiac structures. As, an example, dark blood imaging techniques are advantageous for imaging heart anatomy, delineating scar tissue (e.g., myocardial scar tissue), imaging masses (e.g., lesions) in the heart or other vascularized tissues, imaging vessel walls, imaging vascularized tissues or organs (e.g., liver, pancreas), brain imaging, imaging other arterial and venous structures (e.g., peripheral vessel imaging), and the like. As another example, dark blood imaging techniques are advantageous for distinguishing lesions such as small, early-stage lung cancers from nearby pulmonary vessels or cardiac structures.

The systems and methods described in the present disclosure improve upon previous dark blood imaging techniques by providing a partially unbalanced SSFP pulse sequence, which may be a partially unbalanced gradient-spoiled SSFP pulse sequence. In general, the partially unbalanced SSFP pulse sequence can include an SSFP pulse sequence in which gradients are unbalanced along at least one spatial encoding dimension, and are balanced along at least one other spatial encoding dimension. As a non-limiting example, the partially unbalanced SSFP pulse sequence can include an SSFP pulse sequence having gradients that are unbalanced along the slice select direction, but balanced along the readout direction. Alternatively, the partially unbalanced SSFP sequence can include an SSFP pulse sequence having gradients that are unbalanced along the readout direction, but balanced along the slice select direction. Other combinations of unbalanced and balanced gradients along different spatial encoding directions can also be implemented.

Magnetic resonance data can be acquired as two-dimensional data (e.g., representative of one or more imaging slices) or as three-dimensional data (e.g., representative of an imaging volume, multiple imaging slices, one or more imaging slabs, and so on). In some implementations, the data can be acquired with a Cartesian k-space trajectory. In other implementations, the data can be acquired using non-Cartesian trajectories (e.g., radial, golden angle stack-of-stars, and so on).

Advantageously, the partially unbalanced SSFP pulse sequences can be implemented using interrupted shots (e.g., cardiac-gated shots, respiratory-gated shots, navigator-gated shots, or otherwise discontinuous or interrupted shot patterns) to further reduce motion sensitivity during data acquisition. As one example, the shot duration in the pulse sequence can be set as a fraction of a cardiac cycle, a respiratory cycle, or the like. For instance, the shot duration can be set as less than one-half of the cardiac cycle. As another example, the shot duration can be selected as 1000 ms or less. Additionally or alternatively, the time delay between interrupted shots can be selected (e.g., when the interrupted shots are not gated by some external signal or data). For instance, a time delay of 100 ms or greater can be applied between sequential ones of the interrupted shots.

The partially unbalanced SSFP pulse sequences described in the present disclosure include additional technical improvements relative to previous SSFP pulse sequences, which advantageously reduce motion sensitivity, improve upon previously suboptimal tissue contrast, and improve upon previously poor image quality. As one example, the disclosed partially unbalanced SSFP pulse sequences can include a high sampling bandwidth, which minimizes chemical shift artifacts and enables the use of a very short repetition time (“TR”) and echo time (“TE”). Using a shorter TR maximizes scan efficiency, while using a shorter TE reduces the sensitivity to off-resonance artifacts.

As another example, the disclosed partially unbalanced SSFP pulse sequences can implement a reduced dephasing gradient moment, which minimizes the sensitivity to cardiac and respiratory motion, while providing sufficient spoiling to avoid steady-state image artifacts.

As yet another example, the disclosed partially unbalanced SSFP pulse sequences can include one or more dummy radiofrequency (“RF”) pulses applied immediately before each SSFP echo train, which drives the magnetization into the steady state.

As generally noted above, the disclosed partially unbalanced SSFP pulse sequences can implement interrupted shots. As one example, the interrupted shots can include cardiac-gated shots. Using cardiac gating allows for the readout to be synchronized with the diastolic phase of the cardiac cycle, thereby avoiding image artifacts from flow acceleration and transmitted cardiac pulsations. Techniques for synchronizing data acquisition to one or more phases of the cardiac cycle can include electrocardiographic (“ECG”) gating, pulse gating, and/or utilizing motion-sensitive RF coils.

As another example, the disclosed partially unbalanced SSFP pulse sequences can include the use of a magnetization preparation module, which allows flexible modification of the image contrast. For instance, T2-prepared dark blood SSFP images can be used to characterize abnormalities such as tissue edema caused by inflammation, while T1-prepared dark blood SSFP images can be used for gadolinium-enhanced scans to evaluate the enhancement patterns of tumors. Additionally or alternatively, other magnetization preparations can be implemented in the magnetization preparation module, including RF inversion, RF saturation, T2 preparation, fat suppression, diffusion preparation, arterial spin labeling, or magnetization transfer preparation. In some implementations, a saturation RF pulse, inversion RF pulse, and/or pseudo-continuous spin labeling preparation can be applied to inflowing spins. Magnetic resonance data can also be acquired using dynamic imaging techniques following the administration of a contrast agent, such as gadolinium.

As still another example, the disclosed partially unbalanced SSFP pulse sequences can include a short echo train duration, which reduces the specific absorption rate (“SAR”), maximizes the efficacy of T1-weighted and T2-weighted magnetization preparations, allows the readout to be synchronized to diastole (when cardiac gating is implemented), and minimizes motion sensitivity.

Because of their reduced sensitivity to motion, the systems and methods described in the present disclosure are advantageous for free-breathing MRI, which can be especially helpful for patients who are unable to sustain a breath-hold. In some implementations, the free-breathing imaging can be accompanied with cardiac and/or respiratory gating, which may include navigator gating, or the like. Navigator-gating is advantageous for the acquisition of isotropic data sets with large numbers of thin 3D partitions, which cannot be done within the time-constraints of a breath-hold.

In some implementations, data acquisition can be accelerated using parallel imaging techniques. As one example, data acquisition can be accelerated using a reduced number of phase encoding lines (or other spatial encodings), thereby resulting in an undersampled k-space data set. Parallel imaging reconstruction techniques can then be utilized to reconstruct unaliased images from the undersampled k-space data set. Additionally or alternatively, simultaneous multislice imaging techniques can be implemented to acquire magnetic resonance data from multiple different imaging locations (e.g., imaging slices) at the same time. For instance, a CAIPIRINHA technique can be used to provide simultaneous multislice imaging.

Additionally or alternatively, the partially unbalanced SSFP-echo pulse sequence can be configured to acquire magnetic resonance data using a multi-echo acquisition scheme.

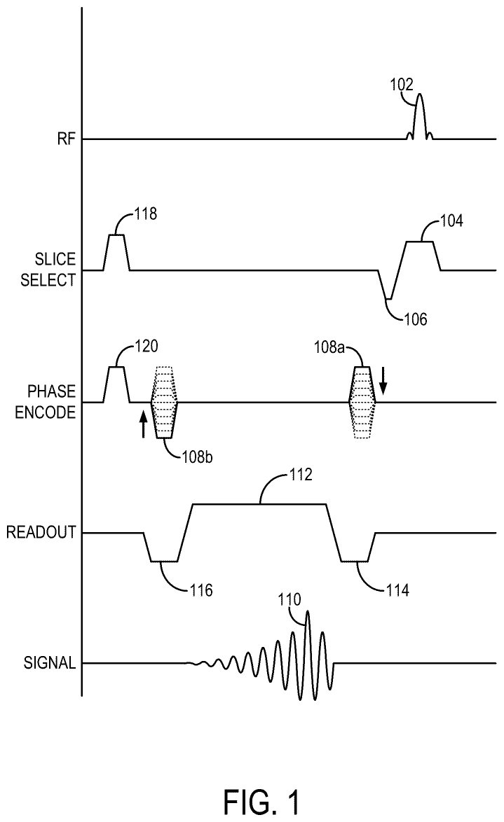

FIG. 1 illustrates an example partially unbalanced time-reversed SSFP (e.g., SSFP-echo) pulse sequence in accordance with some embodiments described in the present disclosure. The pulse sequence shown in FIG. 1 is adapted from an SSFP-echo, or PSIF, pulse sequence, in which a spin-echo signal is refocused from magnetization generated by an RF pulse that was applied during a prior sequence repetition. For instance, in a first shot, or repetition, of the pulse sequence, no magnetic resonance data are acquired. Then, in each subsequent shot, or repetition, of the pulse sequence, magnetic resonance data are acquired by sampling an echo signal that was created by the RF excitation pulse applied in the preceding shot, or repetition. As a result, the partially unbalanced SSFP-echo pulse sequence utilizes an entirely different mechanism for producing dark blood contrast than the more conventional SSFP-FID pulse sequence, in which a free induction decay signal is generated by an RF pulse within the same shot or sequence repetition. For instance, an SSFP-FID pulse sequence generates dark blood contrast through a flip angle-dependent interplay of stimulated echoes, whereas the SSFP-echo pulse sequence creates dark blood contrast through a flip angle-independent flow-dephasing/diffusion mechanism.

In FIG. 1, a single shot, or repetition, of the pulse sequence is illustrated. It will be appreciated by those skilled in the art that the pulse sequence can be repeated for a plurality of different shots or repetitions in order to acquire magnetic resonance data from an imaging volume. In each shot, magnetic resonance data can be acquired from a particular imaging location within a larger imaging volume, which may include an imaging slice, an imaging slab, or the like. As a non-limiting example, in each shot or repetition, one line of k-space data is spatially encoded and acquired. Subsequent repetitions of the pulse sequence sample different lines of k-space for the same imaging location until a prescribed number of lines of k-space data have been acquired, after which data can be acquired from a different imaging location (e.g., a different imaging slice, a different imaging slab) in additional repetitions of the pulse sequence.

It is an advantage of the systems and methods described in the present disclosure that the magnetic resonance data acquired using the SSFP pulse sequence can be acquired using interrupted shots. That is, the subsequent shots or repetitions of the pulse sequence do not need to be continuous in time. With an uninterrupted scan, motion-related dephasing accumulates throughout the entire duration of the scan, rendering it very sensitive to motion-related artifacts. As a result, such scans cannot be reliably used in regions of the body where motion is present, such as the chest or abdomen. By using interrupted shots, motion-related dephasing only accumulates for the duration of each shot, and not for the much longer duration of the scan. As a result, such interrupted scans can reduce motion-related dephasing and associated motion-related artifacts.

As one example, the interrupted shots can include cardiac-gated shots, in which shots of the pulse sequence are initiated based on cardiac signals measured from the subject (e.g., via electrocardiography (“ECG”), or the like). For instance, shots can be initiated based on a particular cardiac phase of the cardiac cycle, such as by synchronizing the readout to the diastolic phase of the cardiac cycle, thereby avoiding image artifacts from flow acceleration and transmitted cardiac pulsations. As another example, the interrupted shots can include respiratory-gated shots, in which shots of the pulse sequence are initiated based on respiratory signals measured from the subject (e.g., via respiratory bellows, or the like). For instance, shots can be initiated based on a particular respiratory phase, such as end-expiration, end-inspiration, or the like. As yet another example, the interrupted shots can include navigator-gated shots, in which shots of the pulse sequence are initiated based on navigator data acquired with the MRI system. Navigator data can be acquired using techniques known in the art, and may include one-dimensional navigator data, two-dimensional navigator data, and/or three-dimensional navigator data.

The pulse sequence includes applying a radio frequency (“RF”) excitation pulse 102. The RF excitation pulse 102 is played out in the presence of a slice-selective gradient 104 in order to produce transverse magnetization in a prescribed imaging slice. Alternatively, slab selection and/or partition encoding can be used for other spatially selective excitations, as is known in the art. The slice-selective gradient 104 includes a rephasing lobe 106 that acts to rephase unwanted phase accruals caused by the RF excitation pulse 102. Following excitation of the nuclear spins in the prescribed imaging slice, a phase encoding gradient 108a and corresponding rewinder gradient 108b is applied to spatially encode a nuclear magnetic resonance signal 110, along one spatial dimension in the prescribed imaging slice. A readout gradient 112 is also applied to spatially encode the signal representative of echo 110 along a second, orthogonal spatial dimension in the prescribed imaging slice. The signal representative of echo 110 is sampled during a data acquisition window. The readout gradient 112 is bridged by a dephasing gradient lobe 114 and a rephasing gradient lobe 116.

As described above, the pulse sequence is repeated and the amplitude and/or polarity of the phase-encoding and rewinder gradients 108a, 108b are stepped through a set of values such that k-space is sampled in a prescribed manner.

In some implementations, the pulse sequence includes adjustable dephasing gradients on the unbalanced gradient axes, such as the adjustable dephasing gradient 118 on the slice select gradient axis and the adjustable dephasing gradient 120 on the phase encoding gradient axis. Including the dephasing gradients on the unbalanced gradient axes helps maintain dark blood image contrast in the magnetic resonance data acquired in subsequent repetitions of the pulse sequence. Such dephasing gradients may be adjustable by adjusting their strength and/or gradient axis on which they are applied. The strength of the dephasing gradients can be a user-selectable parameter; for example, the user can adjust the dephasing gradient strengths via an input on the operator workstation of the MRI system. In general, increasing the strength of the dephasing gradients will increase the motion sensitivity of the pulse sequence.

Additionally or alternatively, the pulse sequence may include one or more magnetization preparation gradients applied along one or more of the gradient axes in order to help establish a steady state magnetization before data acquisition.

In the illustrated example, the magnetic field gradients applied along the slice select direction and the phase encoding direction are unbalanced (i.e., there is a constant unbalanced gradient moment at the end of each sequence repetition along the slice select and phase encoding gradient axes), and the magnetic field gradients applied along the readout direction are balanced. For instance, the area under the readout gradient 112 is equal to the combined area under the dephasing gradient lobe 114 and rephasing gradient lobe 116.

In some implementations, the shot duration of the pulse sequence can be selected to facilitate reducing motion sensitivity in the data acquisition. As a non-limiting example, the shot duration can be selected as a fraction of the cardiac cycle. For instance, the short duration can be selected as one-half of the cardiac cycle.

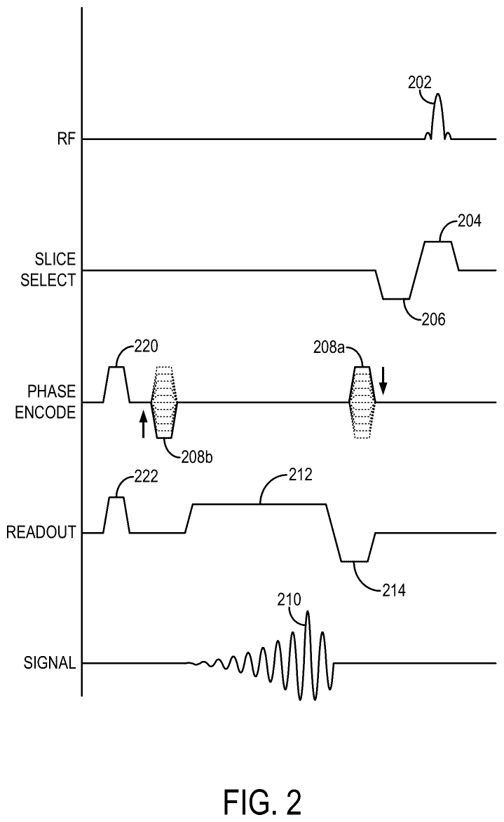

FIG. 2 illustrates another example partially unbalanced SSFP pulse sequence. In this example, the magnetic field gradients are balanced on the slice select gradient axis, and are unbalanced on the phase encoding and readout gradient axes. For instance, the rephasing lobe 206 of the slice-selective gradient 204 is shaped such that the area of the rephasing lobe 206 is equal to the area under the slice-selective gradient 204. As described above, dephasing gradients are applied along the unbalanced gradient axes. Thus, in the example illustrated in FIG. 2, a dephasing gradient 220 is applied along the phase encoding gradient axis, and another dephasing gradient 222 is applied along the readout gradient axis.

The systems and methods described in the present disclosure address the limitations of previous unbalanced gradient-spoiled SSFP techniques by implementing an interrupted shot, partially unbalanced SSFP pulse sequence (e.g., a 2D-SSFP pulse sequence, a 3D-SSFP pulse sequence). The disclosed technique provides for reliable multi-contrast dark blood imaging of the heart, or other anatomical targets.

In an example study, the partially unbalanced SSFP-echo pulse sequences described in the present disclosure were implemented for volumetric multi-contrast dark blood cardiac MRI. An SSFP-echo pulse sequence was used with a balanced readout gradient and unbalanced slice-select gradient, similar to the pulse sequence illustrated in FIG. 1. Scan parameters included: diastolic ECG gating, two shots, 10 dummy RF pulses, sampling bandwidth=1184 Hz/pixel, reconstructed voxel=1.1-mm×1.1-mm×1-mm to 3-mm, adjustable spoiler gradient applied along the slice-select direction, breath-holding or free-breathing with navigator gating. For T1-weighting, a non-selective inversion preparation was applied with the inversion time (“TI”) determined by a TI scout scan. For T2-weighting, a T2prep module was applied with T2prep time of 90 ms and gating to every second heartbeat.

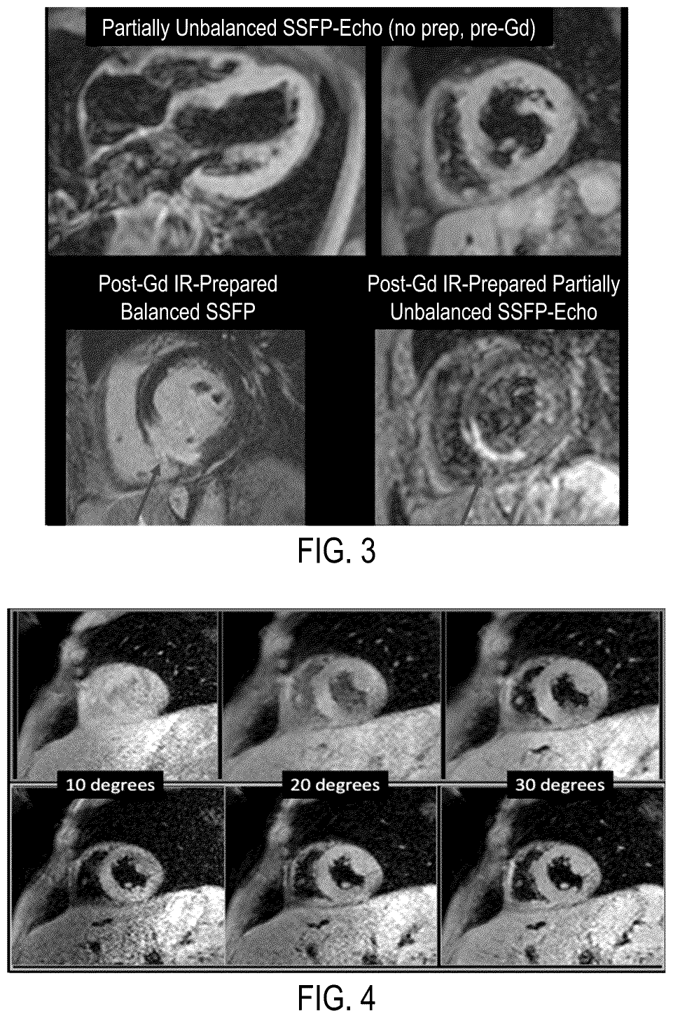

The partially unbalanced SSFP-echo technique was observed to be less sensitive to cardiac motion than previous dark blood SSFP-FID techniques. Regions of late myocardial enhancement were well shown using an inversion preparation, as illustrated in FIG. 3. Navigator gating allowed volumetric imaging of the entire heart as well as T2-weighted dark blood imaging.

ECG-gated partially unbalanced SSFP-echo sequence enables volumetric multi-contrast dark blood imaging of the heart, with less motion sensitivity than previously described fully unbalanced SSFP-FID techniques.

FIG. 4 illustrates an example comparison between images obtained with a fully unbalanced SSFP-FID pulse sequence (top row) and a partially unbalanced SSFP-echo pulse sequence, such as those described in the present disclosure. FIG. 4 further illustrates the differing impact of the excitation flip angle on the intra-cardiac blood pool signal intensity for the imaging techniques. The blood pool appears dark irrespective of flip angle using the partially unbalanced SSFP-echo pulse sequence, whereas the blood pool only appears dark using the fully unbalanced SSFP-FID pulse sequence at large flip angles. Further, whereas the fully unbalanced SSFP-FID sequence shows subtle motion artifacts in the inferolateral segment of the left ventricle, no such motion artifacts are visible with the partially unbalanced SSP-echo sequence.

In another example study, the partially unbalanced SSFP-echo pulse sequences described in the present disclosure were implemented for imaging the lungs. In this example, a free-breathing version of the partially unbalanced SSFP-echo pulse sequence was implemented to enable imaging of the entire chest with 1 mm³ isotropic resolution in less than 5 minutes.

Motion compensation can be obtained using navigator-gating as well as pilot tone respiratory gating, which eliminates the need to directly monitor the diaphragm motion. In a non-limiting example, a golden angle stack-of-stars k-space trajectory was implemented, which has improved motion robustness compared with Cartesian trajectories.

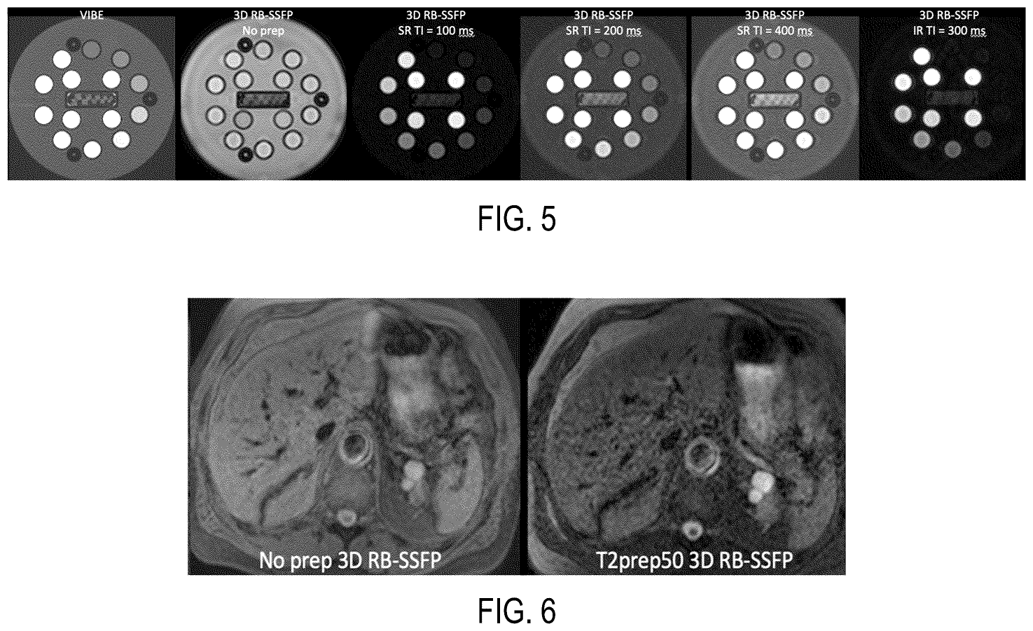

The partially unbalanced SSFP-echo pulse sequences described in the present disclosure can also, advantageously, be adapted to enable multi-contrast imaging. For instance, both T1-weighted and T2-weighted versions of the partially unbalanced SSFP-echo pulse sequences can be implemented. As a non-limiting example, T1-weighted dark blood images can be obtained by applying a saturation or inversion recovery preparation, as illustrated in FIGS. 3 and 5. As another non-limiting example, T2-weighted dark blood images can be obtained by applying an adiabatic T2-preparation, as illustrated in FIG. 6. Advantageously, a partial Fourier readout can be used to shorten the time between a T2-preparation and center of k-space to improve the efficacy of the T2-preparation for T2-weighted imaging implementations.

FIG. 3 illustrates images obtained of a CaliberMRI phantom containing spheres with varying T1 relaxation times. Going clockwise around the outer circle of spheres, the T1 relaxation times progressively increase from 82 msec at the 12 o'clock position to 1724 msec at the 11 o'clock position. The phantom was imaged with VIBE and 3D RB-SSFP (i.e., a partially unbalanced SSFP-echo pulse sequence with unbalanced slice select gradients and balanced readout gradients) using no magnetization preparation (“no prep”), a saturation recovery (“SR”) preparation, or an inversion recovery (“IR”) preparation. The TI represents the time from the magnetization preparation to the center of k-space. Without a T1 preparation, 3D RB-SSFP shows negligible contrast between the spheres, whereas with a magnetization preparation there is a wide range of T1-dependent contrast as a function of the TI and type of preparation (SR vs. IR) used.

FIG. 6 illustrates images obtained from a patient with inferoseptal infarct. The top row of FIG. 6 shows 4-chamber (left) and mid short-axis (right) images of the heart using fat-suppressed 3D RB-SSFP (obtained without a T1-weighted magnetization preparation) provide an artifact-free depiction of the myocardium with uniform dark blood contrast. The bottom row of FIG. 6 shows corresponding mid short-axis late gadolinium-enhanced image acquired using iIR-prepared trueFISP (left). There is transmural delayed enhancement involving the inferoseptal segment of the left ventricle. It is difficult to distinguish the blood pool from the enhancing infarct (arrow) since both appear bright. Late gadolinium-enhanced image (right) acquired using IR-prepared 3D RB-SSFP better shows the extent of the infarct because the blood pool is suppressed.

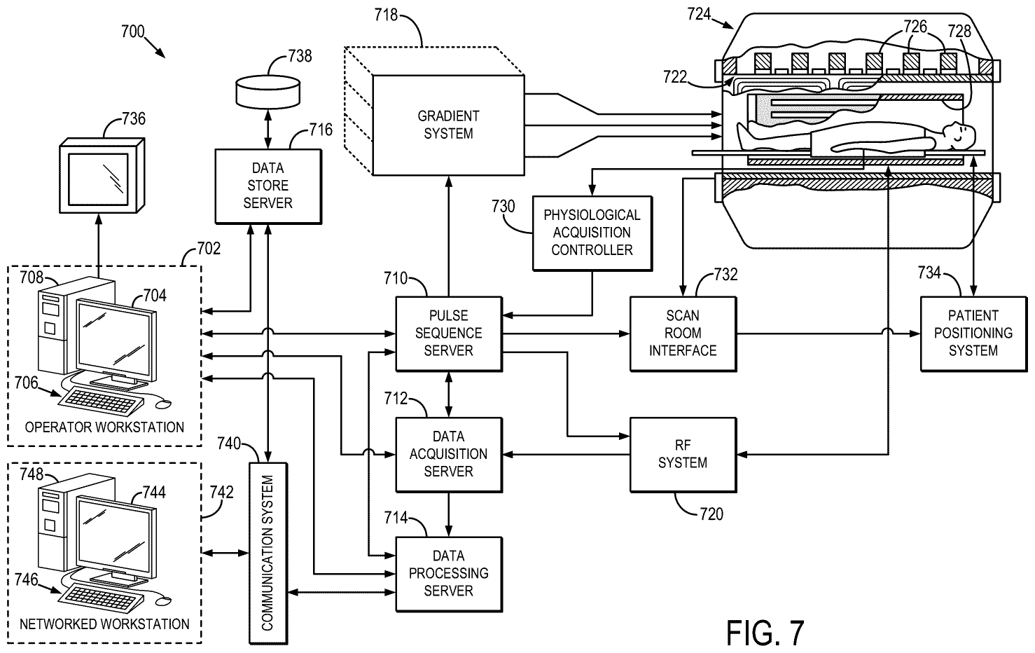

FIG. 7 shows partially unbalanced SSFP-echo images acquired from a patient with left renal cysts (arrow). Contrast between the cysts and other tissues is improved by the application of an adiabatic T2 preparation (using a T2 preparation time of 50 msec). Liver and muscle appear relatively darker, and cerebrospinal fluid relatively brighter, with the T2 preparation.

Referring particularly now to FIG. 7, an example of an MRI system 700 that can implement the methods described here is illustrated. The MRI system 700 includes an operator workstation 702 that may include a display 704, one or more input devices 706 (e.g., a keyboard, a mouse), and a processor 708. The processor 708 may include a commercially available programmable machine running a commercially available operating system. The operator workstation 702 provides an operator interface that facilitates entering scan parameters into the MRI system 700. The operator workstation 702 may be coupled to different servers, including, for example, a pulse sequence server 710, a data acquisition server 712, a data processing server 714, and a data store server 716. The operator workstation 702 and the servers 710, 712, 714, and 716 may be connected via a communication system 740, which may include wired or wireless network connections.

The pulse sequence server 710 functions in response to instructions provided by the operator workstation 702 to operate a gradient system 718 and a radiofrequency (“RF”) system 720. Gradient waveforms for performing a prescribed scan are produced and applied to the gradient system 718, which then excites gradient coils in an assembly 722 to produce the magnetic field gradients Gₓ, G_{y}, and G_{z} that are used for spatially encoding magnetic resonance signals. The gradient coil assembly 722 forms part of a magnet assembly 724 that includes a polarizing magnet 726 and a whole-body RF coil 728.

RF waveforms are applied by the RF system 720 to the RF coil 728, or a separate local coil to perform the prescribed magnetic resonance pulse sequence. Responsive magnetic resonance signals detected by the RF coil 728, or a separate local coil, are received by the RF system 720. The responsive magnetic resonance signals may be amplified, demodulated, filtered, and digitized under direction of commands produced by the pulse sequence server 710. The RF system 720 includes an RF transmitter for producing a wide variety of RF pulses used in MRI pulse sequences. The RF transmitter is responsive to the prescribed scan and direction from the pulse sequence server 710 to produce RF pulses of the desired frequency, phase, and pulse amplitude waveform. The generated RF pulses may be applied to the whole-body RF coil 728 or to one or more local coils or coil arrays.

The RF system 720 also includes one or more RF receiver channels. An RF receiver channel includes an RF preamplifier that amplifies the magnetic resonance signal received by the coil 728 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²};

and the phase of the received magnetic resonance signal may also be determined according to the following relationship:

$$φ = {tan}^{- 1} ( \frac{Q}{I} ) .$$

The pulse sequence server 710 may receive patient data from a physiological acquisition controller 730. By way of example, the physiological acquisition controller 730 may receive signals from a number of different sensors connected to the patient, including electrocardiograph (“ECG”) signals from electrodes, or respiratory signals from a respiratory bellows or other respiratory monitoring devices. These signals may be used by the pulse sequence server 710 to synchronize, or “gate,” the performance of the scan with the subject's heart beat or respiration.

The pulse sequence server 710 may also connect to a scan room interface circuit 732 that receives signals from various sensors associated with the condition of the patient and the magnet system. Through the scan room interface circuit 732, a patient positioning system 734 can receive commands to move the patient to desired positions during the scan.

The digitized magnetic resonance signal samples produced by the RF system 720 are received by the data acquisition server 712. The data acquisition server 712 operates in response to instructions downloaded from the operator workstation 702 to receive the real-time magnetic resonance data and provide buffer storage, so that data is not lost by data overrun. In some scans, the data acquisition server 712 passes the acquired magnetic resonance data to the data processor server 714. In scans that require information derived from acquired magnetic resonance data to control the further performance of the scan, the data acquisition server 712 may be programmed to produce such information and convey it to the pulse sequence server 710. For example, during pre-scans, magnetic resonance data may be acquired and used to calibrate the pulse sequence performed by the pulse sequence server 710. As another example, navigator signals may be acquired and used to adjust the operating parameters of the RF system 720 or the gradient system 718, or to control the view order in which k-space is sampled. In still another example, the data acquisition server 712 may also process magnetic resonance signals used to detect the arrival of a contrast agent in a magnetic resonance angiography (“MRA”) scan. For example, the data acquisition server 712 may acquire magnetic resonance data and processes it in real-time to produce information that is used to control the scan.

The data processing server 714 receives magnetic resonance data from the data acquisition server 712 and processes the magnetic resonance data in accordance with instructions provided by the operator workstation 702. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, calculating motion or flow images, and so on. In some implementations, reconstructed images can be further processed, such as by computing image subtraction or other manipulations or image filtering known in the art.

Images reconstructed by the data processing server 714 are conveyed back to the operator workstation 702 for storage. Real-time images may be stored in a data base memory cache, from which they may be output to operator display 702 or a display 736. Batch mode images or selected real time images may be stored in a host database on disc storage 738. When such images have been reconstructed and transferred to storage, the data processing server 714 may notify the data store server 716 on the operator workstation 702. The operator workstation 702 may be used by an operator to archive the images, produce films, or send the images via a network to other facilities.

The MRI system 700 may also include one or more networked workstations 742. For example, a networked workstation 742 may include a display 744, one or more input devices 746 (e.g., a keyboard, a mouse), and a processor 748. The networked workstation 742 may be located within the same facility as the operator workstation 702, or in a different facility, such as a different healthcare institution or clinic.

The networked workstation 742 may gain remote access to the data processing server 714 or data store server 716 via the communication system 740. Accordingly, multiple networked workstations 742 may have access to the data processing server 714 and the data store server 716. In this manner, magnetic resonance data, reconstructed images, or other data may be exchanged between the data processing server 714 or the data store server 716 and the networked workstations 742, such that the data or images may be remotely processed by a networked workstation 742.

The present disclosure has described one or more preferred embodiments, and it should be appreciated that many equivalents, alternatives, variations, and modifications, aside from those expressly stated, are possible and within the scope of the invention.

1. A method for producing an image of a subject using a magnetic resonance imaging (MRI) system, the method comprising:
(a) acquiring magnetic resonance data from a subject by controlling the MRI system to perform a time-reversed steady-state free precession (SSFP) pulse sequence comprising, in each of a plurality of interrupted shots:
applying a radio frequency (RF) excitation pulse to a prescribed imaging location;
applying unbalanced magnetic field gradients along at least a first spatial encoding direction;
applying balanced magnetic field gradients along at least a second spatial encoding direction; and
(b) reconstructing an image from the magnetic resonance data, wherein the image depicts a dark blood image contrast in which blood is depicted darker than other tissues.

2. The method of claim 1, wherein no magnetic resonance data are acquired in a first one of the plurality of interrupted shots and the magnetic resonance data acquired in each one subsequent one of the plurality interrupted shots are representative of an echo signal created by the RF excitation pulse in a preceding one of the plurality of interrupted shots.

3. The method of claim 1, wherein the first spatial encoding direction is a slice select direction.

4. The method of claim 3, wherein the second spatial encoding direction is a readout direction.

5. The method of claim 1, wherein the first spatial encoding direction is a readout direction.

6. The method of claim 5, wherein the second spatial encoding direction is a slice select direction.

7. The method of claim 1, wherein the magnetic resonance data are spatially encoded along the first spatial encoding direction, the second spatial encoding direction, and a third spatial encoding direction that is orthogonal to the first and second spatial encoding directions, wherein the SSFP pulse sequence further comprises applying unbalanced magnetic field gradients along the third spatial encoding direction.

8. The method of claim 1, wherein the magnetic resonance data are spatially encoded along the first spatial encoding direction, the second spatial encoding direction, and a third spatial encoding direction that is orthogonal to the first and second spatial encoding directions, wherein the SSFP pulse sequence further comprises applying balanced magnetic field gradients along the third spatial encoding direction.

9. The method of claim 1, further comprising acquiring cardiac signal data from the subject, and wherein the plurality of interrupted shots comprises a plurality of cardiac-gated shots, wherein each one of the plurality of cardiac-gated shots is initiated based on the cardiac signal data.

10. The method of claim 9, wherein the cardiac signal data comprise electrocardiography (ECG) data acquired from the subject while the magnetic resonance data are acquired.

11. The method of claim 1, further comprising acquiring respiratory signal data from the subject, and wherein the plurality of interrupted shots comprises a plurality of respiratory-gated shots, wherein each one of the plurality of respiratory-gated shots is initiated based on the respiratory signal data.

12. The method of claim 1, further comprising acquiring navigator data with the MRI system and wherein the plurality of interrupted shots comprises a plurality of navigator-gated shots, wherein each one of the plurality of navigator-gated shots is initiated based on the navigator data.

13. The method of claim 1, wherein the prescribed imaging location comprises an imaging slice.

14. The method of claim 1, wherein the prescribed imaging location comprises an imaging slab.

15. The method of claim 1, wherein each of the plurality of interrupted shots has a shot duration that is selected as a fraction of a cardiac cycle of the subject.

16. The method of claim 15, wherein the shot duration is selected as less than one-half the cardiac cycle.

17. The method of claim 1, wherein each of the plurality of interrupted shots has a shot duration that is less than 1000 ms.

18. The method of claim 1, wherein the pulse sequence further comprises applying magnetization preparation module comprising at least one of RF inversion, RF saturation, T2 preparation, fat suppression, diffusion preparation, arterial spin labeling, or magnetization transfer preparation.

19. The method of claim 1, wherein the time-revered SSFP pulse sequence is a time-reversed gradient-spoiled SSFP pulse sequence.

20. The method of claim 1, further comprising applying a time delay of at least 100 ms between sequential ones of the plurality of interrupted shots.
